SEQUENCE LISTING (2 sequences)

SEQ ID NO: 1 - DNA, 1440 bp, Nicotiana tabacum
  source 1..1440: mol_type = other DNA
atgacggattatagaataccaacgatgactaatatatggagcaatactacatccgatgataatatgatggaagcttttttatcttctgatccgtcgtcgttttggcccggaacaactactacaccaactccccggagttcagtttctccagcgccggcgccggtgacggggattgccggagacccattaaagtctatgccatatttcaaccaagagtcactgcaacagcgactccagactttaatcgatggggctcgcgaagggtggacgtatgccatattttggcaatcgtctgttgtggatttcgcgagcccctcggttttggggtggggagatgggtattataaaggtgaagaagataaaaataagcgtaaaacggcgtcgttttcgcctgactttatcacggaacaagcacaccggaaaaaggttctccgggagctgaattctttaatttccggcacacaaaccggtggtgaaaatgatgctgtagatgaagaagtaactgatactgaatggttttttctgatttccatgacacaatcgtttgttaacggaagcgggcttccgggcctggcgatgtatagttcaagcccgatttgggttactggaacagagagattagctgtttctcactgtgaacgggcccgacaggcccaaggtttcgggcttcagactattgtttgtattccttcagctaatggtgttgttgagctcgggtcaactgagttgatattccagactgctgatttaatgaacaaggttaaagttttgtttaattttaatattgatatgggtgcgactacgggctcaggatcgggctcatgtgctattcaggccgagcccgatccttcagccctttggctgactgatccggcttcttcagttgtggaagtcaaggattcgtcgaatacagttccttcaaggaataccagtaagcaacttgtgtttggaaatgagaattctgaaaatggtaatcaaaattctcagcaaacacaaggatttttcactagggagttgaatttttccgaatatggatttgatggaagtaatactcggtatggaaatgggaatgcgaattcttcgcgttcttgcaagcctgagtctggtgaaatcttgaattttggtgatagtactaagaggagtgcttgcagtgcaaatgggagcttgttttcgggccaatcacagttcgggcccgggcctgcggaggagaacaagaacaagaacaagaaaaggtcacctgcatcaagaggaagcaacgatgaaggaatcctttcatttgtttcgggtgtgattttgccaagttcaaacacggggaagtccggtggaggtggcgattcggatcaatcagatctcgaggcttcggtggtgaaggaggcggatagtagtagagttgtagaccccgagaagaagccgaggaaacgagggaggaaaccggctaac

SEQ ID NO: 2 - DNA, 546 bp, Nicotiana tabacum
  source 1..546: mol_type = other DNA
atgacggattatagaataccaacgatgactaatatatggagcaatactacatccgatgataatatgatggaagcttttttatcttctgatccgtcgtcgttttggcccggaacaactactacaccaactccccggagttcagtttctccagcgccggcgccggtgacggggattgccggagacccattaaagtctatgccatatttcaaccaagagtcactgcaacagcgactccagactttaatcgatggggctcgcaaagggtggacgtatgccatattttggcaatcgtctgttgtggatttcgcgagcccctcggttttggggtggggagatgggtattataaaggtgaagaagataaaaataagcgtaaaacggcgtcgttttcgcctgactttatcacggaacaagcacaccggaaaaaggttctccgggagctgaattctttaatttccggcacacaaaccggtggtgaaaatgatgctgtagatgaagaagtaactgatactgaatggttttatttccatgacacaatcgtttgttaa

(19) United States
(12) Patent Application Publication
(10) Pub. No.: US 2026/0101860 A1
(43) Pub. Date: Apr. 16, 2026

(54) TOBACCO PLANTS WITH REDUCED NICOTINIC ALKALOID LEVELS

(71) Applicant: North Carolina State University, Raleigh, NC (US)

(72) Inventor: Ramsey Lewis, Raleigh, NC (US)

(21) Appl. No.: 19/419,918

(22) Filed: Dec. 15, 2025

Related U.S. Application Data
(63) Continuation of application No. PCT/US2024/033602, filed on Jun. 12, 2024.
(60) Provisional application No. 63/508,735, filed on Jun. 16, 2023.

(51) Int. Cl. A01H 6/82; A01H 1/00; A01H 1/02; A01H 5/12; A24B 15/24

(52) U.S. Cl. CPC A01H 6/823; A01H 1/021; A01H 1/101; A01H 5/12; A24B 15/243

(57) ABSTRACT

The present disclosure provides compositions and methods related to tobacco plants. In particular, the present disclosure provides novel methods for producing tobacco plants, and any related tobacco products, having low nicotinic alkaloid content. Tobacco plants produced according to the methods of the present disclosure exhibit reduced levels of nicotinic alkaloids (e.g., nicotine) compared to both naturally-occurring and transgenic tobacco plants, and thus represent a commercially valuable alternative to currently available tobacco varieties.

### CROSS REFERENCE TO RELATED APPLICATIONS

This application is a continuation of PCT International Patent Application No. PCT/US2024/033602 filed Jun. 12, 2024, which claims priority to and the benefit of U.S. Provisional Patent Application No. 63/508,735 filed Jun. 16, 2023, each of which are incorporated herein by reference in its entirety for all purposes.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 4,741 bytes Byte ASCII (Text) file named “NCSU-42104-601” created on Jun. 12, 2024.

### FIELD

The present disclosure provides compositions and methods related to tobacco plants. In particular, the present disclosure provides novel methods for producing tobacco plants, and any related tobacco products, having low nicotinic alkaloid content. Tobacco plants produced according to the methods of the present disclosure exhibit reduced levels of nicotinic alkaloids (e.g., nicotine) compared to both naturally-occurring and transgenic tobacco plants, and thus represent a commercially valuable alternative to currently available tobacco varieties.

### BACKGROUND

Nicotine is typically the most abundant pyridine alkaloid produced by tobacco, Nicotiana tabacum L., although nornicotine (its demethylated metabolite) can prevail in some plants due to increased activity of genes coding for nicotine demethylase enzymes. Because nicotine contributes to the addictive nature of combustible cigarettes, some public health organizations have recommended the investigation of potentially mandated reduced nicotine levels in combustible cigarettes to reduce human exposure to tobacco smoke-related toxicants (United States Food and Drug Administration, 2018). The World Health Organization (WHO) has recommended that nicotine levels of cigarette tobacco filler be reduced to non-addictive levels of 0.4 mg g⁻¹, or below (WHO, 2015). The U.S. Food and Drug Administration's Center for Tobacco Products has suggested that exclusive exposure to smoke from combustible cigarettes containing tobacco filler in the 0.2-0.7 mg nicotine-per-cigarette range could be associated with reduced addiction potential (U.S. FDA, 2018). Historically, it has been difficult to develop tobacco cultivars that routinely exhibit such ultra-low levels of leaf nicotine accumulation. Thus, there is a need for genetic methodologies that might be used to develop new tobacco cultivars that accumulate nicotine below proposed levels of tolerance.

### SUMMARY

Embodiments of the present disclosure include a tobacco cultivar, or any part thereof, comprising reduced levels of at least one nicotinic alkaloid compared to a corresponding naturally-occurring tobacco plant, or part thereof.

In some embodiments, the cultivar is non-transgenic.

In some embodiments, the at least one nicotinic alkaloid is selected from the group consisting of nicotine, nornicotine, anatabine, and anabasine.

In some embodiments, the at least one nicotinic alkaloid is nicotine, and the cultivar comprises no more than 0.29% nicotine. In some embodiments, the at least one nicotinic alkaloid is nornicotine, and the cultivar comprises no more than 0.02% nornicotine. In some embodiments, the nornicotine is not detectable in the cultivar. In some embodiments, the at least one nicotinic alkaloid is anatabine, and wherein the cultivar comprises no more than 0.01% anatabine. In some embodiments, the anatabine is not detectable in the cultivar. In some embodiments, the at least one nicotinic alkaloid is anabasine, and the cultivar comprises no more than 0.002% anabasine. In some embodiments, the anabasine is not detectable in the cultivar.

In some embodiments, the at least one nicotinic alkaloid is nicotine and nornicotine, and wherein the cultivar comprises no more than 0.29% nicotine and no more than 0.02% nornicotine.

In some embodiments, the cultivar comprises a nic1 allele having reduced expression and/or function compared to wildtype NIC1. In some embodiments, the cultivar comprises a nic2 allele having reduced expression and/or function compared to wildtype NIC2. In some embodiments, the nic1 and/or the nic2 allele is derived from at least one of the following tobacco varieties or lines: LAFC53, LAK326, LATN90, MAFC5, LMAFC34, LAMD 609, Lonibow, Vector 21-41, LA Burley 21, LI Burley 21, and HI Burley 21.

In some embodiments, the cultivar comprises a myc2a allele having reduced expression and/or function compared to wildtype MYC2a. In some embodiments, the myc2a allele comprises at least one nucleotide deletion relative to a wild type MYC2a allele set forth in SEQ ID NO: 1. In some embodiments, the myc2a allele comprises a deletion of one to five nucleotides relative to a wild type MYC2a allele set forth in SEQ ID NO: 1. In some embodiments, the myc2a allele comprises a deletion of five nucleotides relative to a wild type MYC2a allele set forth in SEQ ID NO: 1. In some embodiments, the deletion produces a truncated MYC2A protein. In some embodiments, the myc2a allele comprises a nucleic acid sequence that is at least 70% identical to SEQ ID NO: 2. In some embodiments, the myc2a allele comprises a nucleic acid sequence that is at least 80% identical to SEQ ID NO: 2. In some embodiments, the myc2a allele comprises a nucleic acid sequence that is at least 90% identical to SEQ ID NO: 2. In some embodiments, the myc2a allele is derived from N. tabacum.

In some embodiments, the cultivar further comprises suppression of expression within the cultivar of at least one of BBL (also known as NBB1), A622, quinolate phosphoribosyltransferase (QPT), putrescine N-methyltransferase (PMI), ornithine decarboxylase (ODC), aspartate oxidase (AO), quinolinic acid synthase (QS), N-methylputrescine oxidase (MPO), NtERF221, NtMYC1a, NtMYC1b, or NMYC2b.

In some embodiments, the cultivar is an NCLA161 cultivar, or derived therefrom.

Embodiments of the present disclosure also include a progeny plant, seed, or cell produced from any of the tobacco cultivars described herein.

Embodiments of the present disclosure also include a tobacco product derived from any of the tobacco cultivars, or parts therefrom, described herein. In some embodiments, the product is selected from the group consisting of leaf tobacco, shredded tobacco, cut tobacco, ground tobacco, powder tobacco, tobacco extract, smokeless tobacco, moist or dry snuff, snus, pipe tobacco, cigar tobacco, cigarillo tobacco, cigarette tobacco, and chewing tobacco. In some embodiments, the product is selected from the group consisting of a cigarillo, a cigarette, a kretek cigarette, a filter cigarette, a make-your-own cigarette, a roll-your-own cigarette, a stick or pod for a heated tobacco product, a cigar, tobacco-containing gum, tobacco-containing lozenges, and chewing tobacco.

Embodiments of the present disclosure also include a method of producing a tobacco cultivar comprising reduced levels of at least one nicotinic alkaloid compared to a corresponding naturally-occurring tobacco plant, or part thereof. In accordance with these embodiments, the method includes crossing a first tobacco variety comprising a first low nicotine trait with a second tobacco variety comprising a second low nicotine trait to produce a progeny plant. In some embodiments, the progeny plant comprises a reduced concentration of at least one nicotinic alkaloid, as compared to either parent.

In some embodiments, the method comprises backcrossing.

In some embodiments of the method, the at least one nicotinic alkaloid is nicotine, and the cultivar comprises no more than 0.29% nicotine. In some embodiments of the method, the at least one nicotinic alkaloid is nornicotine, and the cultivar comprises no more than 0.02% nornicotine. In some embodiments of the method, the nornicotine is not detectable in the cultivar. In some embodiments of the method, the at least one nicotinic alkaloid is anatabine, and wherein the cultivar comprises no more than 0.01% anatabine. In some embodiments of the method, the anatabine is not detectable in the cultivar. In some embodiments of the method, the at least one nicotinic alkaloid is anabasine, and the cultivar comprises no more than 0.002% anabasine. In some embodiments of the method, the anabasine is not detectable in the cultivar.

In some embodiments of the method, the first and/or second low nicotine trait comprises a nic1 allele having reduced expression and/or function compared to wildtype NIC. In some embodiments of the method, the first and/or second low nicotine trait comprises a nic2 allele having reduced expression and/or function compared to wildtype NIC2. In some embodiments of the method, the first and/or second tobacco variety comprises a nic1 and/or a nic2 allele derived from at least one of the following tobacco varieties or lines: LAFC53, LAK326, LATN90, MAFC5, LMAFC34, LAMD 609, Lonibow, Vector 21-41, LA Burley 21, LI Burley 21, and HI Burley 21.

In some embodiments of the method, the first and/or second tobacco variety comprises a myc2a allele having reduced expression and/or function compared to wildtype MYC2a. In some embodiments of the method, the myc2a allele comprises at least one nucleotide deletion relative to a wild type MYC2a allele set forth in SEQ ID NO: 1. In some embodiments of the method, the myc2a allele comprises a deletion of one to five nucleotides relative to a wild type MYC2a allele set forth in SEQ ID NO: 1. In some embodiments of the method, the myc2a allele comprises a deletion of five nucleotides relative to a wild type MYC2a allele set forth in SEQ ID NO: 1. In some embodiments of the method, the deletion produces a truncated MYC2A protein. In some embodiments of the method, the myc2a allele comprises a nucleic acid sequence that is at least 70% identical to SEQ ID NO: 2. In some embodiments of the method, the myc2a allele comprises a nucleic acid sequence that is at least 80% identical to SEQ ID NO: 2. In some embodiments of the method, the myc2a allele comprises a nucleic acid sequence that is at least 90% identical to SEQ ID NO: 2. In some embodiments of the method, the myc2a allele is derived from N. tabacum.

In some embodiments of the method, the first low nicotine trait comprises a nic1 and/or nic2 allele having reduced expression and/or function compared to wildtype NIC1 and/or NIC2, and wherein the second low nicotine trait comprises a myc2a allele having reduced expression and/or function compared to wildtype MYC2a.

In some embodiments of the method, the first tobacco variety, the second tobacco variety, and the progeny plant are non-transgenic.

Embodiments of the present disclosure also include a seed or cell obtained from the progeny plant produced according to any of the methods described herein.

Embodiments of the present disclosure also include a tobacco product derived from the progeny plant produced according to any of the methods described herein. In some embodiments, the product is selected from the group consisting of leaf tobacco, shredded tobacco, cut tobacco, ground tobacco, powder tobacco, tobacco extract, smokeless tobacco, moist or dry snuff, snus, pipe tobacco, cigar tobacco, cigarillo tobacco, cigarette tobacco, and chewing tobacco. In some embodiments, the product is selected from the group consisting of a cigarillo, a cigarette, a kretek cigarette, a filter cigarette, a make-your-own cigarette, a roll-your-own cigarette, a stick or pod for a heated tobacco product, a cigar, tobacco-containing gum, tobacco-containing lozenges, and chewing tobacco.

Embodiments of the present disclosure also include a method of producing a Nicotiana tabacum plant having reduced nicotinic alkaloid content. In accordance with these embodiments, the method includes combining in a Nicotiana tabacum plant: (a) one or more genetic modifications that reduces the expression and/or function of MYC2A; and (b) a recessive allele of nic1 and/or a recessive allele of nic2. In some embodiments, the Nicotiana tabacum plant has a nicotinic alkaloid content that is reduced as compared to a corresponding naturally-occurring or non-transformed control tobacco plant.

In some embodiments of the method, the Nicotiana tabacum plant comprises a homozygous recessive allele of nic1 and/or a homozygous recessive allele of nic2.

In some embodiments of the method, the one or more genetic modifications that reduces the expression and/or function of MYC2A is introduced by a Transcription activator-like effector nuclease (TALEN), meganuclease, zinc finger nuclease, a CRISPR/Cas9 system, a CRISPR/Cpf1 system, a CRISPR/Csm1 system, a gene knock-in technique or technology, or any combination thereof.

In some embodiments, the method further comprises suppressing expression within the Nicotiana tabacum plant at least one of BBL (also known as NBB1), A622, quinolate phosphoribosyltransferase (QPI), putrescine N-methyltransferase (PMI), ornithine decarboxylase (ODC), aspartate oxidase (AO), quinolinic acid synthase (QS), N-methylputrescine oxidase (MPO), NtERF221, NtMYC1a, NtMYC1b, or NtMYC2b. In some embodiments, the cultivar is an NCLA161 cultivar, or derived therefrom.

Embodiments of the present disclosure also include a Nicotiana tabacum plant produced by any of the methods described herein. In some embodiments of the method, the plant comprises: (a) one or more genetic modifications that reduces the expression and/or function of MYC2A; and (b) a recessive allele of nic1 and/or a recessive allele of nic2.

Embodiments of the present disclosure also include a progeny plant or seed produced from any of the plants described herein. In some embodiments, the progeny plant or seed comprises: (a) one or more genetic modifications that reduces the expression and/or function of MYC2A; and (b) a recessive allele of nic1 and/or a recessive allele of nic2.

Embodiments of the present disclosure also include a tobacco product comprising tobacco from any of the Nicotiana tabacum plants described herein. In some embodiments, the plant comprises: (a) one or more genetic modifications that reduces the expression and/or function of MYC2A; and (b) a recessive allele of nic1 and/or a recessive allele of nic2.

In some embodiments, the tobacco is selected from the group consisting of leaf tobacco, shredded tobacco, cut tobacco, ground tobacco, powder tobacco, tobacco extract, smokeless tobacco, moist or dry snuff, snus, pipe tobacco, cigar tobacco, cigarillo tobacco, cigarette tobacco, and chewing tobacco. In some embodiments, the product is a reduced-nicotine tobacco product selected from the group consisting of a cigarillo, a cigarette, a kretek cigarette, a filter cigarette, a make-your-own cigarette, a roll-your-own cigarette, a stick or pod for a heated tobacco product, a cigar, snuff, snus, tobacco-containing gum, tobacco-containing lozenges, and chewing tobacco.

### DETAILED DESCRIPTION

Embodiments of the present disclosure include novel methods for producing tobacco plants, and any related tobacco products, having low nicotinic alkaloid content. Tobacco plants produced according to the methods of the present disclosure exhibit reduced levels of nicotinic alkaloids, including nicotine, nornicotine, anatabine and anabasine, compared to both naturally-occurring and transgenic tobacco plants, and thus represent a commercially valuable alternative to currently available tobacco varieties.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term. For example, in some embodiments, it will mean plus or minus 5% of the particular term. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences for the production of a polypeptide, precursor, or RNA (e.g., rRNA, tRNA, sRNA, microRNA, lincRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc.). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, “operably linked” refers to a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous.

As used herein, “gene expression” refers to the biosynthesis or production of a gene product, including the transcription and/or translation of the gene product.

As used herein, the term “oligonucleotide,” refers to a short length of single-stranded polynucleotide chain. Oligonucleotides are typically less than about 300 residues long (e.g., between 15 and 100); however, as used herein, the term is also intended to encompass longer polynucleotide chains. Oligonucleotides are often referred to by their length. For example, a 24-residue oligonucleotide is referred to as a “24-mer.” Oligonucleotides can form secondary and tertiary structures by self-hybridizing or by hybridizing to other polynucleotides. Such structures can include, but are not limited to, duplexes, hairpins, cruciforms, bends, and triplexes.

The term “homology” and “homologous” refers to a degree of identity. There may be partial homology or complete homology. A partially homologous sequence is one that is less than 100% identical to another sequence.

As used herein, the terms “complementary” or “complementarity” are used in reference to polynucleotides (e.g., a sequence of nucleotides such as an oligonucleotide or a target nucleic acid) related by the base-pairing rules. For example, for the sequence “5′-A-G-T-3′ is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary”, “complement”) refer to the nucleotides of a nucleic acid sequence that can bind to another nucleic acid sequence through hydrogen bonds, e.g., nucleotides that are capable of base pairing such as by Watson-Crick base pairing or other base pairing. Nucleotides that can form base pairs, e.g., that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure comprises a “double-stranded nucleic acid”. For example, triplex structures are considered to be “double-stranded.” In some embodiments, any base-paired nucleic acid is a “double-stranded nucleic acid.”

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, “locus” is a chromosome region where a polymorphic nucleic acid, trait determinant, gene, or marker is located. The loci of this disclosure comprise one or more polymorphisms in a population; e.g., alternative alleles are present in some individuals. As used herein, “allele” refers to an alternative nucleic acid sequence at a particular locus. The length of an allele can be as small as 1 nucleotide base, but is typically larger. For example, a first allele can occur on one chromosome, while a second allele occurs on a second homologous chromosome, e.g., as occurs for different chromosomes of a heterozygous individual, or between different homozygous or heterozygous individuals in a population. As used herein, the term “chromosome interval” designates a contiguous linear span of genomic DNA that resides on a single chromosome.

As used herein, “introgression” or “introgress” refers to the transmission of a desired allele of a genetic locus from one genetic background to another.

As used herein, “crossed” or “cross” means to produce progeny via fertilization (e.g., cells, seeds or plants) and includes crosses between plants (sexual) and self-fertilization (selfing).

As used herein, “backcross” and “backcrossing” refer to the process whereby a progeny plant is repeatedly crossed back to one of its parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. The initial cross gives rise to the F1 generation. The term “BC1” refers to the second use of the recurrent parent, “BC2” refers to the third use of the recurrent parent, and so on. In some aspects, a backcross is performed repeatedly, with a progeny individual of each successive backcross generation being itself backcrossed to the same parental genotype.

As used herein, “single gene converted” or “single gene conversion” refers to plants that are developed using a plant breeding technique known as backcrossing, or via genetic engineering, wherein essentially all of the desired morphological and physiological characteristics of a variety are recovered in addition to the single gene transferred into the variety via the backcrossing technique or via genetic engineering.

As used herein, the term “variety” refers to a population of plants that share constant characteristics which separate them from other plants of the same species. A variety is often, although not always, sold commercially. While possessing one or more distinctive traits, a variety is further characterized by a very small overall variation between individuals within that variety. A “pure line” variety may be created by several generations of self-pollination and selection, or vegetative propagation from a single parent using tissue or cell culture techniques. A variety can be essentially derived from another line or variety. As defined by the International Convention for the Protection of New Varieties of Plants (Dec. 2, 1961, as revised at Geneva on Nov. 10, 1972; on Oct. 23, 1978; and on Mar. 19, 1991), a variety is “essentially derived” from an initial variety if: a) it is predominantly derived from the initial variety, or from a variety that is predominantly derived from the initial variety, while retaining the expression of the essential characteristics that result from the genotype or combination of genotypes of the initial variety; b) it is clearly distinguishable from the initial variety; and c) except for the differences which result from the act of derivation, it conforms to the initial variety in the expression of the essential characteristics that result from the genotype or combination of genotypes of the initial variety. Essentially derived varieties can be obtained, for example, by the selection of a natural or induced mutant, a somaclonal variant, a variant individual from plants of the initial variety, backcrossing, or transformation. A first tobacco variety and a second tobacco variety from which the first variety is essentially derived, are considered as having essentially identical genetic background. A “line” as distinguished from a variety most often denotes a group of plants used non-commercially, for example in plant research. A line typically displays little overall variation between individuals for one or more traits of interest, although there may be some variation between individuals for other traits.

As used herein, “selecting” or “selection” in the context of marker-assisted selection or breeding refer to the act of picking or choosing desired individuals, normally from a population, based on certain pre-determined criteria.

As used herein, the term “trait” refers to one or more detectable characteristics of a cell or organism which can be influenced by genotype. The phenotype can be observable to the naked eye, or by any other means of evaluation known in the art, e.g., microscopy, biochemical analysis, genomic analysis, an assay for a particular disease tolerance, etc. In some cases, a phenotype is directly controlled by a single gene or genetic locus, e.g., a “single gene trait.” In other cases, a phenotype is the result of several genes.

As used herein, the terms “genetic mutation” or “genetic alteration” refers to an inheritable genetic modification introduced into a gene to alter the expression or activity of a product encoded by the gene. Such a modification can be in any sequence region of a gene, for example, in a promoter, 5′ UTR, exon, intron, 3′ UTR, or terminator region. In an aspect, a mutation reduces, inhibits, or eliminates the expression or activity of a gene product. In another aspect, a mutation increases, elevates, strengthens, or augments the expression or activity of a gene product. In an aspect, mutations are not natural polymorphisms that exist in a particular tobacco variety or cultivar. As used herein, a “mutant allele” refers to an allele from a locus where the allele comprises a mutation. As used herein, “mutagenic” refers to generating a mutation without involving a transgene or with no mutation-related transgene remaining in an eventual mutant. In an aspect, mutagenic is cisgenic. In another aspect, mutagenic is via gene or genome editing. In a further aspect, mutagenic is via random mutagenesis, for example, chemical (e.g., EMS) or physical (r-irradiation) mutagenesis.

As used herein, “polymorphism” means the presence of one or more variations in a population. A polymorphism may manifest as a variation in the nucleotide sequence of a nucleic acid or as a variation in the amino acid sequence of a protein. Polymorphisms include the presence of one or more variations of a nucleic acid sequence or nucleic acid feature at one or more loci in a population of one or more individuals. The variation may comprise but is not limited to one or more nucleotide base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism (RFLP), and a tag SNP. A genetic marker, a gene, a DNA-derived sequence, an RNA-derived sequence, a promoter, a 5′ untranslated region of a gene, a 3′ untranslated region of a gene, microRNA, siRNA, a tolerance locus, a satellite marker, a transgene, mRNA, ds mRNA, a transcriptional profile, and a methylation pattern may also comprise polymorphisms. In addition, the presence, absence, or variation in copy number of the preceding may comprise polymorphisms.

The term “plant” as used herein encompasses a whole plant, a grafted plant, ancestor(s) and progeny of the plants and plant parts, including seeds, shoots, stems, roots (including tubers), rootstock, scion, and plant cells, tissues and organs. The plant may be in any form including suspension cultures, embryos, meristematic regions, callus tissue, leaves, gametophytes, sporophytes, pollen, and microspores. Plants that are particularly useful in the methods of the present disclosure include all plants which belong to the Nicotiana family.

As used herein, the term “tobacco” refers to any plant in the Nicotiana genus that produces nicotinic alkaloids. Tobacco also refers to products comprising material produced by a Nicotiana plant, and therefore includes, for example, cured tobacco, tobacco strips (destemmed tobacco), cut tobacco, expanded tobacco, reconstituted tobacco, cigarettes, cigars, chewing tobacco and forms of smokeless tobacco such as snuff and snus. The nicotine content of reconstituted tobacco is less than the nicotine content of the tobacco used to produce reconstituted tobacco due to the non-tobacco components contained in finished reconstituted tobacco sheet. Expanded tobacco has greater filling power than cut tobacco in a cigarette due to the increased volume of expanded tobacco. Examples of Nicotiana species include but are not limited to the following: Nicotiana acaulis, Nicotiana acuminata, Nicotiana acuminata var. multiflora, Nicotiana africana, Nicotiana alata, Nicotiana amplexicaulis, Nicotiana arentsii, Nicotiana attenuata, Nicotiana benavidesii, Nicotiana benthamiana, Nicotiana bigelovii, Nicotiana bonariensis, Nicotiana cavicola, Nicotiana clevelandii, Nicotiana cordifolia, Nicotiana corymbosa, Nicotiana debneyi, Nicotiana excelsior, Nicotiana forgetiana, Nicotiana fragrans, Nicotiana glauca, Nicotiana glutinosa, Nicotiana goodspeedii, Nicotiana gossei, Nicotiana hybrid, Nicotiana ingulba, Nicotiana kawakamii, Nicotiana knightiana, Nicotiana langsdorffii, Nicotiana linearis, Nicotiana longiflora, Nicotiana maritima, Nicotiana megalosiphon, Nicotiana miersii, Nicotiana noctiflora, Nicotiana nudicaulis, Nicotiana obtusifolia, Nicotiana occidentalis, Nicotiana occidentalis subsp. hesperis, Nicotiana otophora, Nicotiana paniculata, Nicotiana pauciflora, Nicotiana petunioides, Nicotiana plumbaginifolia, Nicotiana quadrivalvis, Nicotiana raimondii, Nicotiana repanda, Nicotiana rosulata, Nicotiana rosulata subsp. ingulba, Nicotiana rotundifolia, Nicotiana rustica, Nicotiana setchellii, Nicotiana simulans, Nicotiana solanifolia, Nicotiana spegazzinii, Nicotiana stocktonii, Nicotiana suaveolens, Nicotiana sylvestris, Nicotiana tabacum, Nicotiana thyrsiflora, Nicotiana tomentosa, Nicotiana tomentosiformis, Nicotiana trigonophylla, Nicotiana umbratica, Nicotiana undulata, Nicotiana velutina, Nicotiana wigandioides, and Nicotiana x sanderae.

As used herein, the term “transgenic plant” refers to a plant that comprises a nucleic acid sequence that also is present per se in another organism or species or that is optimized, relative to host codon usage, from another organism or species. Both monocotyledonous and dicotyledonous angiosperm or gymnosperm plant cells may be transformed in various ways known to the art. For example, see Klein et al., Biotechnology 4: 583-590 (1993); Bechtold et al., C. R. Acad. Sci. Paris 316:1194-1199 (1993); Bent et al., Mol. Gen. Genet. 204:383-396 (1986); Paszowski et al., EMBO J. 3: 2717-2722 (1984); Sagi et al., Plant Cell Rep. 13: 262-266 (1994).

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, plant biology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Low Nicotine Tobacco Plants

Tobacco alkaloid accumulation is considered to be a complex trait influenced by the numerous genes and the environment. Researchers have historically investigated the use of allelic variability at the NIC1 and NIC2 loci (also referred to as the A and B loci) to achieve lower nicotine levels in tobacco. The NIC2 locus has been found to consist of a series of genes on N. tabacum linkage group 19 that encode for Ethylene Response Factor (ERF) transcription factors that globally influence the expression of structural genes in the tobacco alkaloid biosynthesis pathway. This cluster of genes was found to be deleted in ‘LA Burley 21’ (a backcross-derived, nic1/nic1 nic2/nic2 version of Burley 21). A similar array of genes resides at or near the Nic1 locus on N. tabacum linkage group 7, where an epigenetically silenced allele of ERF199 is believed to underlie the effect on alkaloid accumulation at this locus in LA Burley 21. Such variability has been used to develop low alkaloid breeding lines such as LA Burley 21 and LAFC53. However, materials homozygous for the recessive alleles at the NIC1 and NIC2 loci, alone, do not routinely produce cured leaf with average nicotine levels of below 0.4 mg g⁻¹.

Embodiments of the present disclosure include a tobacco cultivar development method for generating novel tobacco genotypes in which genetic variability at the NIC1 and NIC2 loci derived from LAFC53 (Chaplin, 1975) is combined with a mutated allele of the N. tabacum gene Myc2a that encodes for a transcription factor that positively regulates expression of genes in the nicotine biosynthetic pathway. A deleterious 5 bp mutation in Myc2a was previously identified in Tobacco Introduction TI 313 of the U.S. Nicotiana Germplasm Collection (Burner et al., 2022). Surprisingly and unexpectedly, the homozygous combination of the nic1 and nic2 alleles from LAFC53 and the mutated myc2a allele from TI 313 exhibits significantly lower nicotine levels than tobacco genotypes homozygous for the nic1 and nic2 from LAFC53 alone. This suggests that adding a mutant myc2a allele further suppresses global expression of nicotine biosynthetic genes over that produced by the recessive nic1 and nic2 alleles alone. The results of this approach are tobacco genotypes that produce some of the lowest reported nicotine levels for tobacco plants, along with corresponding reductions in anabasine, anatabine, and nornicotine.

In accordance with the above, embodiments of the present disclosure include a tobacco cultivar, or any part thereof, that comprises reduced levels of at least one nicotinic alkaloid compared to a corresponding naturally-occurring tobacco plant, or part thereof. In some embodiments, the tobacco cultivars produced according to the methods of the present disclosure includes at least one nicotinic alkaloid is selected from the group consisting of nicotine, nornicotine, anatabine, and anabasine. In some embodiments, the cultivar is non-transgenic.

In some embodiments, the tobacco cultivars of the present disclosure include reduced levels of nicotine. In some embodiments, the tobacco cultivar comprises no more than 0.30% nicotine. In some embodiments, the tobacco cultivar comprises no more than 0.29% nicotine. In some embodiments, the tobacco cultivar comprises no more than 0.28% nicotine. In some embodiments, the tobacco cultivar comprises no more than 0.27% nicotine. In some embodiments, the tobacco cultivar comprises no more than 0.26% nicotine. In some embodiments, the tobacco cultivar comprises no more than 0.25% nicotine. In some embodiments, the tobacco cultivar comprises no more than 0.24% nicotine. In some embodiments, the tobacco cultivar comprises no more than 0.23% nicotine. In some embodiments, the tobacco cultivar comprises no more than 0.22% nicotine. In some embodiments, the tobacco cultivar comprises no more than 0.21% nicotine. In some embodiments, the tobacco cultivar comprises no more than 0.20% nicotine. In some embodiments, the tobacco cultivar comprises no more than 0.19% nicotine. In some embodiments, the tobacco cultivar comprises no more than 0.18% nicotine. In some embodiments, the tobacco cultivar comprises no more than 0.17% nicotine. In some embodiments, the tobacco cultivar comprises no more than 0.16% nicotine. In some embodiments, the tobacco cultivar comprises no more than 0.15% nicotine. In some embodiments, the tobacco cultivar comprises no more than 0.14% nicotine. In some embodiments, the tobacco cultivar comprises no more than 0.13% nicotine. In some embodiments, the tobacco cultivar comprises no more than 0.12% nicotine. In some embodiments, the tobacco cultivar comprises no more than 0.11% nicotine. In some embodiments, the tobacco cultivar comprises no more than 0.10% nicotine. In some embodiments, the tobacco cultivar comprises no more than 0.09% nicotine. In some embodiments, the tobacco cultivar comprises no more than 0.08% nicotine. In some embodiments, the tobacco cultivar comprises no more than 0.07% nicotine. In some embodiments, the tobacco cultivar comprises no more than 0.06% nicotine. In some embodiments, the tobacco cultivar comprises no more than 0.05% nicotine. In some embodiments, the tobacco cultivar comprises no more than 0.04% nicotine. In some embodiments, the tobacco cultivar comprises no more than 0.03% nicotine. In some embodiments, the tobacco cultivar comprises no more than 0.02% nicotine. In some embodiments, the tobacco cultivar comprises no more than 0.01% nicotine.

In some embodiments, the tobacco cultivar comprises from about 0.1% to about 0.30% nicotine. In some embodiments, the tobacco cultivar comprises from about 0.1% to about 0.25% nicotine. In some embodiments, the tobacco cultivar comprises from about 0.1% to about 0.20% nicotine. In some embodiments, the tobacco cultivar comprises from about 0.1% to about 0.15% nicotine. In some embodiments, the tobacco cultivar comprises from about 0.15% to about 0.30% nicotine. In some embodiments, the tobacco cultivar comprises from about 0.20% to about 0.30% nicotine. In some embodiments, the tobacco cultivar comprises from about 0.25% to about 0.30% nicotine. In some embodiments, the tobacco cultivar comprises from about 0.15% to about 0.25% nicotine. In some embodiments, the tobacco cultivar comprises from about 0.1% to about 0.20% nicotine. In some embodiments, the tobacco cultivar comprises from about 0.1% to about 0.29% nicotine. In some embodiments, the tobacco cultivar comprises from about 0.15% to about 0.29% nicotine. In some embodiments, the tobacco cultivar comprises from about 0.20% to about 0.29% nicotine. In some embodiments, the tobacco cultivar comprises from about 0.25% to about 0.29% nicotine.

In some embodiments, the tobacco cultivars of the present disclosure include reduced levels of nornicotine. In some embodiments, the cultivar comprises no more than 0.02% nornicotine. In some embodiments, the cultivar comprises no more than 0.01% nornicotine. In some embodiments, the cultivar comprises no more than 0.009% nornicotine. In some embodiments, the cultivar comprises no more than 0.008% nornicotine. In some embodiments, the cultivar comprises no more than 0.007% nornicotine. In some embodiments, the cultivar comprises no more than 0.006% nornicotine. In some embodiments, cultivar comprises no more than 0.005% nornicotine. In some embodiments, the cultivar comprises from about 0.005% to about 0.01% nornicotine. In some embodiments, the cultivar comprises from about 0.006% to about 0.01% nornicotine. In some embodiments, the cultivar comprises from about 0.007% to about 0.01% nornicotine. In some embodiments, the cultivar comprises from about 0.008% to about 0.01% nornicotine. In some embodiments, the cultivar comprises from about 0.009% to about 0.01% nornicotine. In some embodiments, the cultivar comprises from about 0.005% to about 0.009% nornicotine. In some embodiments, the cultivar comprises from about 0.005% to about 0.008% nornicotine. In some embodiments, the cultivar comprises from about 0.005% to about 0.007% nornicotine. In some embodiments, the cultivar comprises from about 0.005% to about 0.006% nornicotine. In some embodiments, nornicotine is not detectable, or is beyond the limits of detection, in the cultivar.

In some embodiments, the tobacco cultivars of the present disclosure include reduced levels of anatabine. In some embodiments, the cultivar comprises no more than 0.02% anatabine. In some embodiments, the cultivar comprises no more than 0.01% anatabine. In some embodiments, the cultivar comprises no more than 0.009% anatabine. In some embodiments, the cultivar comprises no more than 0.008% anatabine. In some embodiments, the cultivar comprises no more than 0.007% anatabine. In some embodiments, the cultivar comprises no more than 0.006% anatabine. In some embodiments, cultivar comprises no more than 0.005% anatabine. In some embodiments, the cultivar comprises from about 0.005% to about 0.01% anatabine. In some embodiments, the cultivar comprises from about 0.006% to about 0.01% anatabine. In some embodiments, the cultivar comprises from about 0.007% to about 0.01% anatabine. In some embodiments, the cultivar comprises from about 0.008% to about 0.01% anatabine. In some embodiments, the cultivar comprises from about 0.009% to about 0.01% anatabine. In some embodiments, the cultivar comprises from about 0.005% to about 0.009% anatabine. In some embodiments, the cultivar comprises from about 0.005% to about 0.008% anatabine. In some embodiments, the cultivar comprises from about 0.005% to about 0.007% anatabine. In some embodiments, the cultivar comprises from about 0.005% to about 0.006% anatabine. In some embodiments, anatabine is not detectable, or is beyond the limits of detection, in the cultivar.

In some embodiments, the tobacco cultivars of the present disclosure include reduced levels of anabasine. In some embodiments, the cultivar comprises no more than 0.002% anabasine. In some embodiments, the cultivar comprises no more than 0.001% anabasine. In some embodiments, the cultivar comprises no more than 0.0009% anabasine. In some embodiments, the cultivar comprises no more than 0.0008% anabasine. In some embodiments, the cultivar comprises no more than 0.0007% anabasine. In some embodiments, the cultivar comprises no more than 0.0006% anatabine. In some embodiments, cultivar comprises no more than 0.0005% anabasine. In some embodiments, cultivar comprises no more than 0.0004% anabasine. In some embodiments, cultivar comprises no more than 0.0003% anabasine. In some embodiments, cultivar comprises no more than 0.0002% anabasine. In some embodiments, cultivar comprises no more than 0.0001% anabasine. In some embodiments, the cultivar comprises from about 0.0004% to about 0.002% anabasine. In some embodiments, the cultivar comprises from about 0.0005% to about 0.002% anabasine. In some embodiments, the cultivar comprises from about 0.0006% to about 0.002% anabasine. In some embodiments, the cultivar comprises from about 0.0007% to about 0.002% anabasine. In some embodiments, the cultivar comprises from about 0.0008% to about 0.002% anabasine. In some embodiments, the cultivar comprises from about 0.0009% to about 0.002% anabasine. In some embodiments, the cultivar comprises from about 0.001% to about 0.002% anabasine. In some embodiments, the cultivar comprises from about 0.0015% to about 0.002% anabasine. In some embodiments, the cultivar comprises from about 0.0004% to about 0.0015% anabasine. In some embodiments, the cultivar comprises from about 0.0004% to about 0.008% anabasine. In some embodiments, the cultivar comprises from about 0.0005% to about 0.0015% anabasine. In some embodiments, the cultivar comprises from about 0.0008% to about 0.0012% anabasine. In some embodiments, anabasine is not detectable, or is beyond the limits of detection, in the cultivar.

In some embodiments, the cultivar comprises a nic1 allele having reduced expression and/or function compared to wildtype NICL. In some embodiments, the cultivar comprises a nic2 allele having reduced expression and/or function compared to wildtype NIC2. In some embodiments, the cultivar comprises a nic1 allele and a nic2 allele having reduced expression and/or function compared to wildtype NIC1 and wildtype NIC2 alleles. In some embodiments, the nic1 and/or the nic2 allele is derived from at least one of the following varieties or lines: LAFC53, LAK326, LATN90, MAFC5, LMAFC34, LAMD 609, Lonibow, Vector 21-41, LA Burley 21, LI Burley 21, and HI Burley 21. In some embodiments, the first and/or second tobacco variety comprises a nic1 and/or nic2 null allele (deletion). In some embodiments, the nic1 and/or nic2 null allele is derived from other N. tabacum germplasm.

In some embodiments, the cultivar comprises a myc2a allele having reduced expression and/or function compared to wildtype MYC2a. In some embodiments, the myc2a allele comprises at least one nucleotide deletion relative to a wild type MYC2a allele set forth in SEQ ID NO: 1. In some embodiments, the myc2a allele comprises a deletion of one to five nucleotides relative to a wild type MYC2a allele set forth in SEQ ID NO: 1. In some embodiments, the myc2a allele comprises a deletion of five nucleotides relative to a wild type MYC2a allele set forth in SEQ ID NO: 1. In some embodiments, the deletion produces a truncated MYC2A protein. In some embodiments, the myc2a allele comprises a nucleic acid sequence that is at least 70% identical to SEQ ID NO: 2. In some embodiments, the myc2a allele comprises a nucleic acid sequence that is at least 75% identical to SEQ ID NO: 2. In some embodiments, the myc2a allele comprises a nucleic acid sequence that is at least 80% identical to SEQ ID NO: 2. In some embodiments, the myc2a allele comprises a nucleic acid sequence that is at least 85% identical to SEQ ID NO: 2. In some embodiments, the myc2a allele comprises a nucleic acid sequence that is at least 90% identical to SEQ ID NO: 2. In some embodiments, the myc2a allele comprises a nucleic acid sequence that is at least 95% identical to SEQ ID NO: 2. In some embodiments, the myc2a allele comprises a nucleic acid sequence that is at least 98% identical to SEQ ID NO: 2. In some embodiments, the myc2a allele is derived from N. tabacum.

In some embodiments, the tobacco cultivars of the present disclosure are non-transgenic (e.g., do not possess a heterologous transgene). In accordance with these embodiments, the tobacco cultivars of the present disclosure can be produced through the breeding (e.g., backcrossing) of various tobacco lines with desired trait(s) corresponding to reduced levels of at least one nicotinic alkaloid. As such, the tobacco cultivars produced according to any of the embodiments of the present disclosure are not naturally-occurring.

As would be understood by one of ordinary skill in the art based on the present disclosure, the tobacco cultivars having reduced levels of at least one nicotinic alkaloid as described herein can also be produced using transgenic approaches. In some embodiments, the tobacco cultivars of the present disclosure can be engineered to include one or more traits corresponding to reduced levels of at least one nicotinic alkaloid. For example, tobacco plants can be engineered to include nic1 and/or nic2 alleles substantially similar to those found in LAFC53, LAK326, LATN90, MAFC5, LMAFC34, LAMD 609, Lonibow, Vector 21-41, LA Burley 21, LI Burley 21, and HI Burley 21. These nic1 and/or nic2 alleles can have one or more genetic alterations that result in reduced levels of at least one nicotinic alkaloid (e.g., deletion, truncation, loss-of-function, or hylomorphic mutations). Additionally, tobacco plants can be engineered to include a myc2a allele that is substantially similar to that found in TI 313. This myc2a allele can have one or more genetic alterations that result in reduced levels of at least one nicotinic alkaloid (e.g., deletion, truncation, loss-of-function, or hylomorphic mutations). Such genetic alterations can be engineered using any means known in the art, including but not limited to, Transcription activator-like effector nucleases (TALENs), meganuclease, zinc finger nuclease, and a clustered regularly-interspaced short palindromic repeats (CRISPR)/Cas9 system, a CRISPR/Cpf1 system, a CRISPR/Csm1 system, or any combination thereof (see, e.g., Gaj et al., Trends in Biotechnology, 31(7):397-405 (2013).

In some embodiments, the present technology provides a method for producing a tobacco plant having reduced nicotinic alkaloid content, the method comprising combining in a tobacco plant (e.g., Nicotiana tabacum): (a) a genetic modification that reduces the expression and/or function of MYC2A as compared to a corresponding naturally-occurring or non-transformed control tobacco plant; and (b) a recessive allele of nic1 and/or a recessive allele of nic2.

In some embodiments, introducing a recessive allele of nic1 and/or a recessive allele nic2 can comprise incorporating one or more of the recessive alleles via conventional breeding into, for example, a tobacco plant (e.g., Nicotiana tabacum) comprising one or more genetic modifications that reduces the expression and/or function of MYC2A in the tobacco plant as compared to a wildtype tobacco plant.

In some embodiments, a tobacco plant produced by the methods of the present technology having (a) a genetic modification that reduces the expression and/or function of MYC2A; and (b) a recessive allele of nic1 and/or a recessive allele of nic2 may comprise a nicotinic alkaloid content that is reduced by at least about 40% (e.g., at least about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, or more, or any range or value therein) as compared to a corresponding naturally-occurring or non-transformed control tobacco plant. In some embodiments, the nicotinic alkaloid that is reduced in the tobacco plant may be nicotine, wherein the nicotine content may be reduced by about 90% or more (e.g., about 90%, 91%, 92%, 93%, 94%, 95%, 96%, 97%, 98%, 99% or more) as compared to a corresponding naturally-occurring or non-transformed control tobacco plant.

In some embodiments, the tobacco plant produced by the methods of the present technology having (a) a genetic modification that reduces the expression and/or function of MYC2A; and (b) a recessive allele of nic1 and/or a recessive allele of nic2 comprises reduced levels of nicotine. In some embodiments, the tobacco cultivar comprises no more than 0.29% nicotine. In some embodiments, the tobacco cultivar comprises no more than 0.28% nicotine. In some embodiments, the tobacco cultivar comprises no more than 0.27% nicotine. In some embodiments, the tobacco cultivar comprises no more than 0.26% nicotine. In some embodiments, the tobacco cultivar comprises no more than 0.25% nicotine. In some embodiments, the tobacco cultivar comprises no more than 0.24% nicotine. In some embodiments, the tobacco cultivar comprises no more than 0.23% nicotine. In some embodiments, the tobacco cultivar comprises no more than 0.22% nicotine. In some embodiments, the tobacco cultivar comprises no more than 0.21% nicotine. In some embodiments, the tobacco cultivar comprises no more than 0.20% nicotine. In some embodiments, the tobacco cultivar comprises no more than 0.19% nicotine. In some embodiments, the tobacco cultivar comprises no more than 0.18% nicotine. In some embodiments, the tobacco cultivar comprises no more than 0.17% nicotine. In some embodiments, the tobacco cultivar comprises no more than 0.16% nicotine. In some embodiments, the tobacco cultivar comprises no more than 0.15% nicotine. In some embodiments, the tobacco cultivar comprises no more than 0.14% nicotine. In some embodiments, the tobacco cultivar comprises no more than 0.13% nicotine. In some embodiments, the tobacco cultivar comprises no more than 0.12% nicotine. In some embodiments, the tobacco cultivar comprises no more than 0.11% nicotine. In some embodiments, the tobacco cultivar comprises no more than 0.10% nicotine. In some embodiments, the tobacco cultivar comprises no more than 0.09% nicotine. In some embodiments, the tobacco cultivar comprises no more than 0.08% nicotine. In some embodiments, the tobacco cultivar comprises no more than 0.07% nicotine. In some embodiments, the tobacco cultivar comprises no more than 0.06% nicotine. In some embodiments, the tobacco cultivar comprises no more than 0.05% nicotine. In some embodiments, the tobacco cultivar comprises no more than 0.04% nicotine. In some embodiments, the tobacco cultivar comprises no more than 0.03% nicotine. In some embodiments, the tobacco cultivar comprises no more than 0.02% nicotine. In some embodiments, the tobacco cultivar comprises no more than 0.01% nicotine.

In some embodiments, the tobacco cultivar comprises from about 0.1% to about 0.30% nicotine. In some embodiments, the tobacco cultivar comprises from about 0.1% to about 0.25% nicotine. In some embodiments, the tobacco cultivar comprises from about 0.1% to about 0.20% nicotine. In some embodiments, the tobacco cultivar comprises from about 0.1% to about 0.15% nicotine. In some embodiments, the tobacco cultivar comprises from about 0.15% to about 0.30% nicotine. In some embodiments, the tobacco cultivar comprises from about 0.20% to about 0.30% nicotine. In some embodiments, the tobacco cultivar comprises from about 0.25% to about 0.30% nicotine. In some embodiments, the tobacco cultivar comprises from about 0.15% to about 0.25% nicotine. In some embodiments, the tobacco cultivar comprises from about 0.1% to about 0.20% nicotine. In some embodiments, the tobacco cultivar comprises from about 0.1% to about 0.29% nicotine. In some embodiments, the tobacco cultivar comprises from about 0.15% to about 0.29% nicotine. In some embodiments, the tobacco cultivar comprises from about 0.20% to about 0.29% nicotine. In some embodiments, the tobacco cultivar comprises from about 0.25% to about 0.29% nicotine.

In some embodiments, the tobacco plant produced by the methods of the present technology having (a) a genetic modification that reduces the expression and/or function of MYC2A; and (b) a recessive allele of nic1 and/or a recessive allele of nic2 comprises reduced levels of nornicotine. In some embodiments, the plant comprises no more than about 0.02% nornicotine, no more than about 0.01% nornicotine, or no more than about 0.005% nornicotine. In some embodiments, the plant comprises from about 0.005% to about 0.02% nornicotine, or from about 0.01% to about 0.02% nornicotine. In some embodiments, nornicotine is not detectable, or is beyond the limits of detection, in the tobacco.

In some embodiments, the tobacco plant produced by the methods of the present technology having (a) a genetic modification that reduces the expression and/or function of MYC2A; and (b) a recessive allele of nic1 and/or a recessive allele of nic2 comprises reduced levels of anatabine. In some embodiments, the plant comprises no more than 0.01% anatabine. In some embodiments, the plant comprises from about 0.001% to about 0.01% anatabine, from about 0.002% to about 0.01% anatabine, from about 0.003% to about 0.01% anatabine, from about 0.004% to about 0.01% anatabine, from about 0.005% to about 0.01% anatabine, from about 0.006% to about 0.01% anatabine, from about 0.007% to about 0.01% anatabine, from about 0.008% to about 0.01% anatabine, or from about 0.009% to about 0.01% anatabine. In some embodiments, anatabine is not detectable, or is beyond the limits of detection, in the tobacco plant.

In some embodiments, the tobacco plant produced by the methods of the present technology having (a) a genetic modification that reduces the expression and/or function of MYC2A; and (b) a recessive allele of nic1 and/or a recessive allele of nic2 comprises reduced levels of anabasine. In some embodiments, the plant comprises from about 0.0004% to about 0.002% anabasine, from about 0.0004% to about 0.0015% anabasine, from about 0.0004% to about 0.008% anabasine, from about 0.0005% to about 0.0015% anabasine, and from about 0.0008% to about 0.0012% anabasine. In some embodiments, anabasine is not detectable, or is beyond the limits of detection, in the tobacco plant.

As described herein, the genetic modifications can be engineered using any means known in the art, including but not limited to, Transcription activator-like effector nucleases (TALENs), meganuclease, zinc finger nuclease, a CRISPR/Cas9 system, a CRISPR/Cpf1 system, a CRISPR/Csm1 system, a gene knock-in technique or technology, or any combination thereof.

For example, in some embodiments, the methods of the present technology relate to the use of a CRISPR/Cas system that binds to a target site in a region of interest in a genome, wherein the CRISPR/Cas system comprises a CRISPR/Cas nuclease and an engineered crRNA/tracrRNA (or single guide RNA (sgRNA) or guide RNA (gRNA)). In some embodiments, the CRISPR system generally comprises (i) a polynucleotide encoding a Cas protein, and (ii) at least one sgRNA for RNA-guided genome engineering in plant cells. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Cys3, Cse1, Cse2, Csc1, Csc2, Csa5, Csn2, Csm2, Csm3, Csm4, Csm5, Csm6, Smr1, Cmr3, Cmr4, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In some embodiments, the Cas protein is a Streptococcuspyogenes Cas9 protein. These enzymes are known. For example, the amino acid sequence of S. pyogenes Cas9 protein may be found in the SwissProt database under accession number Q99ZW2. In some embodiments, the methods of the present technology relate to the use of a CRISPR/Cpf1 system that binds to a target site in a region of interest in a genome. In some embodiments, the methods of the present technology relate to the use of a CRISPR/Csm1 system that binds to a target site in a region of interest in a genome.

In some embodiments, the CRISPR/Cas, CRISPR/Cpf1, or CRISPR/Csm1 system recognizes a target site in myc2a. In some embodiments, the CRISPR/Cas, CRISPR/Cpf1, or CRISPR/Csm1 system generates a specific sequence change in myc2a, such as a mutation resulting in a deletion of one or more nucleotides, and/or a nucleotide substitution, which results in reduced activity/expression of MYC2A (e.g., deletion, truncation, loss-of-function, or hylomorphic mutations). In some embodiments, the CRISPR/Cas, CRISPR/Cpf1, or CRISPR/Csm1 system generates a specific sequence change via gene knock-in or gene replacement. Methods of gene knock-in or gene replacement are well-known in the art. CRISPR-based methods of gene knock-in or gene replacement can utilize homology-directed repair (HDR) mechanisms, non-homologous end joining (NHEJ) mechanisms, or both HDR and NHEJ mechanisms. A non-limiting example of a CRISPR-based method utilizing both HDR and NHEJ mechanisms is called tandem repeat-HDR (TR-HDR), described in Lu et al., “Targeted, efficient sequence insertion and replacement in rice,” Nature Biotechnology, 38(12):1402-1407. doi: 10.1038/s41587-020-0581-5, (2020), which is incorporated herein by reference in its entirety.

In some embodiments, the methods described herein employ a meganuclease DNA binding domain for binding to a region of interest in the genome of a plant cell. Meganucleases are engineered versions of naturally-occurring restriction enzymes that typically have extended DNA recognition sequences (e.g., about 14 to about 40 base pairs in length). Meganucleases (also known as homing endonucleases) are commonly grouped into five families based on sequence and structure motifs: the LAGLIDADG family, the GIY-YIG family, the His-Cyst box family, the PD-(D/E)XK family, and the HNH family. In some embodiments, the meganuclease comprises an engineered homing endonuclease. The recognition sequences of homing endonucleases and meganucleases such as I-Sce, I-CeuI, PI-PspI, PI-Sce, I-SceIV, I-CsmI, I-PanI, I-SceII, I-PpoI, I-SceIII, I-CreI, I-TevI, I-TevII, and I-TevIII are known.

In some embodiments, the meganuclease is tailored to recognize a target in a myc2a allele. In some embodiments, the meganuclease generates a specific sequence change in the myc2a allele, such as a mutation resulting in a deletion of one or more nucleotides, and/or a nucleotide substitution, which results in reduced activity/expression of MYC2A (e.g., deletion, truncation, loss-of-function, or hylomorphic mutations).

In some embodiments, the methods described herein employ transcription activator-like effector nucleases (TALENs) to edit plant genomes by inducing double-strand breaks (DSBs). TALENs are restriction enzymes that can be engineered to cleave specific sequences of DNA. TALENs are constructed by fusing a TAL effector DNA-binding domain to a DNA cleavage domain (e.g., a nuclease domain such as that derived from the FokI endonuclease). Transcription activator-like effectors (TALEs) can be engineered according to methods known in the art to bind to a desired DNA sequence, and when combined with a nuclease, provide a technique for cutting DNA at specific locations. For example, in some embodiments, the use of TALEN technology generates a specific sequence change in a myc2a allele, such as a mutation resulting in a deletion of one or more nucleotides, and/or a nucleotide substitution, which results in reduced activity/expression of MYC2A (e.g., deletion, truncation, loss-of-function, or hylomorphic mutations).

In some embodiments, the compositions and methods described herein employ zinc finger nucleases (ZFNs) to edit plant genomes by inducing double-strand breaks (DSBs). ZFNs are artificial restriction enzymes generated by fusing a zinc finder DNA-binding domain to a DNA cleavage domain (e.g., a nuclease domain such as that derived from the FokI endonuclease). ZFNs can be engineered to bind and cleave DNA at specific locations. ZFNs contain two protein domains. The first domain is the DNA-binding domain, which contains eukaryotic transcription factors and the zinc finger. The second domain is a nuclease domain that contains the FokI restriction enzyme responsible for cleaving DNA. ZFNs can be engineered according to methods known in the art to bind to a desired DNA sequence and cleave DNA at specific locations. For example, after a target sequence in a nicotine biosynthesis gene is identified, a corresponding ZFN sequence is engineered and inserted into a plasmid. The plasmid is inserted into a target cell where it is translated to produce a functional ZFN, which then enters the nucleus where it binds to and cleaves its target sequence introducing a double strand break (DSB). Such an approach can be employed to introduce an exogenous DNA sequence into the target gene as the DSB is being repaired through either homology-directed repair or non-homologous end-joining. For example, in some embodiments, the use of ZFN technology generates a specific sequence change in a myc2a allele, such as a mutation resulting in a deletion of one or more nucleotides, and/or a nucleotide substitution, which results in reduced activity/expression of MYC2A (e.g., deletion, truncation, loss-of-function, or hylomorphic mutations).

In some embodiments, the present technology further comprises suppressing the expression of an endogenous gene encoding a transcription factor that positively regulates alkaloid production such as the NtERF221, NtMYC1a, NtMYC1b, and/or NtMYC2b gene to decrease nicotinic alkaloid levels in a plant.

In some embodiments, the present technology further comprises suppressing the expression of one or more nicotinic alkaloid biosynthesis genes such as the BBL (also known as NBB1), A622, QPT (quinolate phosphoribosyltransferase), PMT (putrescine methyltransferase), ODC (ornithine decarboxylase), AO (aspartate oxidase), QS (quinolinic acid synthase), and MPO (N-methylputrescine oxidase) gene to decrease nicotinic alkaloid levels in a plant.

Examples of methods that may be used for suppressing an ERF199, an ERF189, an NtERF221, an NtMYC1a, an NtMYC1b, an NtMYC2b, a BBL, an A622, a QPT, a PMT, an ODC, an AO, a QS, and/or an MPO gene include, but are not limited to, antisense, sense co-suppression, RNAi, artificial microRNA, virus-induced gene silencing (VIGS), antisense, sense co-suppression, targeted mutagenesis, and/or targeted genome engineering methods including, but not limited to, Transcription activator-like effector nucleases (TALENs), meganuclease, zinc finger nuclease, and a clustered regularly-interspaced short palindromic repeats (CRISPR)/Cas9 system, a CRISPR/Cpf1 system, a CRISPR/Csm1 system, or any combination thereof.

In accordance with the above, embodiments of the present disclosure also include a progeny plant, seed, or cell produced from any of the tobacco cultivars described herein, produced using transgenic and/or non-transgenic methods. Embodiments of the present disclosure also include a tobacco product derived from any of the tobacco cultivars, or parts therefrom, described herein. In some embodiments, the tobacco product is selected from the group consisting of leaf tobacco, shredded tobacco, cut tobacco, ground tobacco, powder tobacco, tobacco extract, smokeless tobacco, moist or dry snuff, snus, pipe tobacco, cigar tobacco, cigarillo tobacco, cigarette tobacco, and chewing tobacco. In some embodiments, the tobacco product is selected from the group consisting of a cigarillo, a cigarette, a kretek cigarette, a filter cigarette, a make-your-own cigarette, a roll-your-own cigarette, a stick or pod for a heated tobacco product, a cigar, tobacco-containing gum, tobacco-containing lozenges, and chewing tobacco.

Embodiments of the present disclosure also include a method of producing a tobacco cultivar comprising reduced levels of at least one nicotinic alkaloid compared to a corresponding naturally-occurring tobacco plant, or part thereof. In accordance with these embodiments, the method includes crossing a first tobacco variety comprising a first low nicotine trait with a second tobacco variety comprising a second low nicotine trait to produce a progeny plant. In some embodiments, the progeny plant comprises a reduced concentration of at least one nicotinic alkaloid compared to either the first or second tobacco variety. In some embodiments, the method comprises backcrossing. Such tobacco cultivars having reduced levels of at least one nicotinic alkaloid can be produced using transgenic and/or non-transgenic methods, as would be recognized by one of ordinary skill in the art.

In some embodiments, and as described further above, the at least one nicotinic alkaloid is selected from the group consisting of nicotine, nornicotine, anatabine, and anabasine. In some embodiments, the at least one nicotinic alkaloid is nicotine, and wherein the cultivar comprises no more than 0.30% nicotine. In some embodiments, the at least one nicotinic alkaloid is nornicotine, and wherein the cultivar comprises no more than 0.02% nornicotine. In some embodiments, the at least one nicotinic alkaloid is anatabine, and wherein the cultivar comprises no more than 0.01% anatabine. In some embodiments, the at least one nicotinic alkaloid is anabasine, and wherein the cultivar comprises no more than 0.002% anabasine. In some embodiments, the at least one nicotinic alkaloid is nicotine and nornicotine, and wherein the cultivar comprises no more than 0.30% nicotine and no more than 0.02% nornicotine.

In some embodiments, and as described further above, the at least one nicotinic alkaloid is selected from the group consisting of nicotine, nornicotine, anatabine, and anabasine. In some embodiments, the at least one nicotinic alkaloid is nicotine, and wherein the cultivar comprises no more than 0.29% nicotine. In some embodiments, the at least one nicotinic alkaloid is nornicotine, and wherein the cultivar comprises no more than 0.02% nornicotine. In some embodiments, the at least one nicotinic alkaloid is anatabine, and wherein the cultivar comprises no more than 0.01% anatabine. In some embodiments, the at least one nicotinic alkaloid is anabasine, and wherein the cultivar comprises no more than 0.002% anabasine. In some embodiments, the at least one nicotinic alkaloid is nicotine and nornicotine, and wherein the cultivar comprises no more than 0.29% nicotine and no more than 0.02% nornicotine.

In some embodiments, and as described further above, the at least one nicotinic alkaloid is selected from the group consisting of nicotine, nornicotine, anatabine, and anabasine. In some embodiments, the at least one nicotinic alkaloid is nicotine, and wherein the cultivar comprises no more than 0.25% nicotine. In some embodiments, the at least one nicotinic alkaloid is nornicotine, and wherein the cultivar comprises no more than 0.02% nornicotine. In some embodiments, the at least one nicotinic alkaloid is anatabine, and wherein the cultivar comprises no more than 0.01% anatabine. In some embodiments, the at least one nicotinic alkaloid is anabasine, and wherein the cultivar comprises no more than 0.002% anabasine. In some embodiments, the at least one nicotinic alkaloid is nicotine and nornicotine, and wherein the cultivar comprises no more than 0.25% nicotine and no more than 0.02% nornicotine.

In some embodiments, the first and/or second low nicotine trait comprises a nic1 allele having reduced expression and/or function compared to wildtype NICL. In some embodiments, the first and/or second low nicotine trait comprises a nic2 allele having reduced expression and/or function compared to wildtype NIC2. In some embodiments, the first and/or second tobacco variety comprises a nic1 and/or a nic2 allele derived from at least one of the following varieties or lines: LAFC53, LAK326, LATN90, MAFC5, LMAFC34, LAMD 609, Lonibow, Vector 21-41, LA Burley 21, LI Burley 21, and HI Burley 21. In some embodiments, the first and/or second tobacco variety comprises a nic1 and/or nic2 null allele (deletion). In some embodiments, the nic1 and/or nic2 null allele is derived from other N. tabacum germplasm. In some embodiments, the first and/or second tobacco variety comprises a myc2a allele having reduced expression and/or function compared to wildtype MYC2a. In some embodiments, the myc2a allele is derived from N. tabacum.

In some embodiments of the method, the first low nicotine trait comprises a nic1 and/or nic2 allele having reduced expression and/or function compared to wildtype NIC1 and/or NIC2, and wherein the second low nicotine trait comprises a myc2a allele having reduced expression and/or function compared to wildtype MYC2a. In some embodiments of the method, the first tobacco variety, the second tobacco variety, and the progeny plant are non-transgenic.

Embodiments of the present disclosure also include a seed or cell obtained from the progeny plant produced according to any of the methods described herein.

Embodiments of the present disclosure also include a tobacco product derived from the progeny plant produced according to any of the methods described herein. In some embodiments, the product is selected from the group consisting of leaf tobacco, shredded tobacco, cut tobacco, ground tobacco, powder tobacco, reconstituted tobacco, tobacco extract, smokeless tobacco, moist or dry snuff, snus, pipe tobacco, cigar tobacco, cigarillo tobacco, cigarette tobacco, and chewing tobacco. In some embodiments, the product is selected from the group consisting of a cigarillo, a cigarette, a kretek cigarette, a filter cigarette, a make-your-own cigarette, a roll-your-own cigarette, a stick or pod for a heated tobacco product, a cigar, snuff, snus, tobacco-containing gum, tobacco-containing lozenges, and chewing tobacco. Make-your-own cigarettes are produced with empty cigarette tubes and a table-top injector machine which injects tobacco filler into the empty cigarette tube. Roll-your-own cigarettes are made by hand from rolling typical, flat rolling papers. Tobacco heating devices, also known as heat-not-burn products or heated tobacco products, are electronic devices that heat tobacco below the point of combustion in a pod or stick of the device which results in an aerosol (without smoke) available for inhalation. Commercial examples include IQOS® and glo®.

Embodiments of the present disclosure also include a method of producing a Nicotiana tabacum plant having reduced nicotinic alkaloid content. In accordance with these embodiments, the method includes combining in a Nicotiana tabacum plant: (a) one or more genetic modifications that reduces the expression and/or function of MYC2A; and (b) a recessive allele of nic1 and/or a recessive allele of nic2. In some embodiments, the Nicotiana tabacum plant has a nicotinic alkaloid content that is reduced as compared to a corresponding naturally-occurring or non-transformed control tobacco plant. In some embodiments of the method, the Nicotiana tabacum plant comprises a homozygous recessive allele of nic1 and/or a homozygous recessive allele of nic2.

In some embodiments of the method, the one or more genetic modifications that reduces the expression and/or function of MYC2A is introduced by a Transcription activator-like effector nuclease (TALEN), meganuclease, zinc finger nuclease, a CRISPR/Cas9 system, a CRISPR/Cpf1 system, a CRISPR/Csm1 system, a gene knock-in technique or technology, or any combination thereof.

In some embodiments, the method further comprises suppressing expression within the Nicotiana tabacum plant of at least one of BBL (also known as NBB1), A622, quinolate phosphoribosyltransferase (QP7), putrescine N-methyltransferase (PMT), ornithine decarboxylase (ODC), aspartate oxidase (AO), quinolinic acid synthase (QS), N-methylputrescine oxidase (MPO), NtERF221, NtMYC1a, NtMYC1b, or NtMYC2b. In some embodiments, the cultivar is an NCLA161 cultivar, or derived therefrom.

Embodiments of the present disclosure also include a Nicotiana tabacum plant produced by of the methods described herein. In some embodiments of the method, the plant comprises: (a) one or more genetic modifications that reduces the expression and/or function of MYC2A; and (b) a recessive allele of nic1 and/or a recessive allele of nic2.

Embodiments of the present disclosure also include a progeny plant or seed produced from any of the plants described herein. In some embodiments, the progeny plant or seed comprises: (a) one or more genetic modifications that reduces the expression and/or function of MYC2A; and (b) a recessive allele of nic1 and/or a recessive allele of nic2.

Embodiments of the present disclosure also include a tobacco product comprising tobacco from any of the Nicotiana tabacum plants described herein. In some embodiments, the plant comprises: (a) one or more genetic modifications that reduces the expression and/or function of MYC2A; and (b) a recessive allele of nic1 and/or a recessive allele of nic2.

In some embodiments, the tobacco is selected from the group consisting of leaf tobacco, shredded tobacco, cut tobacco, ground tobacco, powder tobacco, tobacco extract, smokeless tobacco, moist or dry snuff, snus pipe tobacco, cigar tobacco, cigarillo tobacco, cigarette tobacco, and chewing tobacco. In some embodiments, the product is a reduced-nicotine tobacco product selected from the group consisting of a cigarillo, a cigarette, a kretek cigarette, a filter cigarette, a make-your-own cigarette, a roll-your-own cigarette, a stick or pod for a heated tobacco product, a cigar, snuff, snus, tobacco-containing gum, tobacco-containing lozenges, and chewing tobacco.

Sequences. The nucleic acid sequences below are referenced throughout the descriptions in the present disclosure.

Wild type myc2a (K326):
(SEQ ID NO: 1)
ATGACGGATTATAGAATACCAACGATGACTAATATATGGAGCA
ATACTACATCCGATGATAATATGATGGAAGCTTTTTTATCTTC
TGATCCGTCGTCGTTTTGGCCCGGAACAACTACTACACCAACT
CCCCGGAGTTCAGTTTCTCCAGCGCCGGCGCCGGTGACGGGGA
TTGCCGGAGACCCATTAAAGTCTATGCCATATTTCAACCAAGA
GTCACTGCAACAGCGACTCCAGACTTTAATCGATGGGGCTCGC
GAAGGGTGGACGTATGCCATATTTTGGCAATCGTCTGTTGTGG
ATTTCGCGAGCCCCTCGGTTTTGGGGTGGGGAGATGGGTATTA
TAAAGGTGAAGAAGATAAAAATAAGCGTAAAACGGCGTCGTTT
TCGCCTGACTTTATCACGGAACAAGCACACCGGAAAAAGGTTC
TCCGGGAGCTGAATTCTTTAATTTCCGGCACACAAACCGGTGG
TGAAAATGATGCTGTAGATGAAGAAGTAACTGATACTGAATGG
TTTTTTCTGATTTCCATGACACAATCGTTTGTTAACGGAAGCG
GGCTTCCGGGCCTGGCGATGTATAGTTCAAGCCCGATTTGGGT
TACTGGAACAGAGAGATTAGCTGTTTCTCACTGTGAACGGGCC
CGACAGGCCCAAGGTTTCGGGCTTCAGACTATTGTTTGTATTC
CTTCAGCTAATGGTGTTGTTGAGCTCGGGTCAACTGAGTTGAT
ATTCCAGACTGCTGATTTAATGAACAAGGTTAAAGTTTTGTTT
AATTTTAATATTGATATGGGTGCGACTACGGGCTCAGGATCGG
GCTCATGTGCTATTCAGGCCGAGCCCGATCCTTCAGCCCTTTG
GCTGACTGATCCGGCTTCTTCAGTTGTGGAAGTCAAGGATTCG
TCGAATACAGTTCCTTCAAGGAATACCAGTAAGCAACTTGTGT
TTGGAAATGAGAATTCTGAAAATGGTAATCAAAATTCTCAGCA
AACACAAGGATTTTTCACTAGGGAGTTGAATTTTTCCGAATAT
GGATTTGATGGAAGTAATACTCGGTATGGAAATGGGAATGCGA
ATTCTTCGCGTTCTTGCAAGCCTGAGTCTGGTGAAATCTTGAA
TTTTGGTGATAGTACTAAGAGGAGTGCTTGCAGTGCAAATGGG
AGCTTGTTTTCGGGCCAATCACAGTTCGGGCCCGGGCCTGCGG
AGGAGAACAAGAACAAGAACAAGAAAAGGTCACCTGCATCAAG
AGGAAGCAACGATGAAGGAATCCTTTCATTTGTTTCGGGTGTG
ATTTTGCCAAGTTCAAACACGGGGAAGTCCGGTGGAGGTGGCG
ATTCGGATCAATCAGATCTCGAGGCTTCGGTGGTGAAGGAGGC
GGATAGTAGTAGAGTTGTAGACCCCGAGAAGAAGCCGAGGAAA
CGAGGGAGGAAACCGGCTAAC.
Truncated myc2a (TI 313 from Burner et al.
2022):
(SEQ ID NO: 2)
ATGACGGATTATAGAATACCAACGATGACTAATATATGGAGCA
ATACTACATCCGATGATAATATGATGGAAGCTTTTTTATCTTC
TGATCCGTCGTCGTTTTGGCCCGGAACAACTACTACACCAACT
CCCCGGAGTTCAGTTTCTCCAGCGCCGGCGCCGGTGACGGGGA
TTGCCGGAGACCCATTAAAGTCTATGCCATATTTCAACCAAGA
GTCACTGCAACAGCGACTCCAGACTTTAATCGATGGGGCTCGC
AAAGGGTGGACGTATGCCATATTTTGGCAATCGTCTGTTGTGG
ATTTCGCGAGCCCCTCGGTTTTGGGGTGGGGAGATGGGTATTA
TAAAGGTGAAGAAGATAAAAATAAGCGTAAAACGGCGTCGTTT
TCGCCTGACTTTATCACGGAACAAGCACACCGGAAAAAGGTTC
TCCGGGAGCTGAATTCTTTAATTTCCGGCACACAAACCGGTGG
TGAAAATGATGCTGTAGATGAAGAAGTAACTGATACTGAATGG
TTTTATTTCCATGACACAATCGTTTGTTAA.

### 3. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Given the possibility for mandated reduced nicotine levels in conventional cigarettes to below so-called “sub-threshold levels of addiction” in various countries around the world (including the United States), there is commercial interest in tobacco cultivars that would produce cured leaf with nicotine levels that would allow manufacturers to achieve target nicotine levels. Currently, nicotine levels of cigarette tobacco filler of 0.04% to 0.08% are being discussed and/or recommended by regulatory agencies. An elite flue-cured tobacco cultivar having good agronomic performance that routinely accumulates nicotine at such low levels (when grown under systems of conventional agronomic management and averaged over all stalk positions) is not currently available to the tobacco industry.

Thus, embodiments of the present disclosure include low nicotine content of a newly established tobacco hybrid, NCLA161 (nic1 and nic2 alleles derived from LAFC53 combined with the mutated myc2a allele from N. tabacum). The source of the genetic variability at the NIC1 and NIC2 loci that influences low nicotine content was derived from flue-cured tobacco breeding line LAFC53 (Chaplin, 1975). The source of the mutant allele myc2a was Tobacco Introduction TI 313 (Burner et al., 2022). The backcross breeding method (Fehr, 1987) was used to combine the nic1 and nic2 alleles from LAFC53 and the mutant myc2a allele derived from TI 313 in triple homozygous condition in the elite genetic background of flue-cured tobacco cultivar “K326.” Six generations of backcrossing were carried out, followed by two generations of self-pollination to establish a triple homozygous stable BC₆F₃ line designated as NCLA161. The NCLA161 cultivar is non-transgenic.

More specifically, five plants of each of NCLA161, K326, and K326 22 (a previously developed nearly-isogenic version of K326 into which the recessive nic1 and nic2 alleles were backcrossed) were transplanted to 3.79 liter pots in a greenhouse. The experimental design for the greenhouse experiment was a randomized complete block design and the bottom 1 to 2 inches of the pots were submerged in 5 gallon buckets containing water for subirrigation purposes. In order to stimulate alkaloid biosynthesis, plants were topped (removal of apical inflorescence or apical meristem) when approximately 50% of plants were in flower. Lateral meristems (suckers) were removed by hand from leaf axils approximately every other day post-topping until leaf harvest. The top two leaves from each plant were harvested 21 days after topping, treated with ethephon according to manufacturer's recommendations (Arysta LifeScience, Cary, NC), air-cured, and ground to pass through a 1 mm sieve. Alkaloid profiles were determined using gas chromatography according to previously described methodology (Lewis et al. 2015).

Mean nicotine content of NCLA161 was found to be ˜1% of that for K326 and −40% of that for K326 22 (Table 1), thus indicating a desirable combinatory effect of novel genotypes containing mutations in nic1, nic2, and myc2a on lowering nicotine content in tobacco leaves. Concentrations of nornicotine, anabasine, and anatabine for NCLA161 were non-detectable as compared to those observed for K326per se.

TABLE 1
Average alkaloid content for top two leaves of three tobacco genotypes evaluated
in greenhouse experiments (“nd” indicates non-detectable levels).

| Greenhouse | | Percent Dry Weight (%) | | | |
|---|---|---|---|---|---|
| Plant | Genotype | Nicotine | Nornicotine | Anabasine | Anatabine |
| 1 | K326 | 1.0178 | 0.0210 | 0.0030 | 0.0223 |
| 2 | K326 | 0.9634 | 0.0218 | 0.0033 | 0.0298 |
| 3 | K326 | 0.6951 | 0.0161 | 0.0028 | 0.0162 |
| 4 | K326 | 1.1838 | 0.0245 | 0.0046 | 0.0262 |
| 5 | K326 | 0.6548 | 0.0132 | 0.0026 | 0.0202 |
| | Average | 0.9029 | 0.0193 | 0.0033 | 0.0230 |
| 6 | K326 22 | 0.0084 | nd | nd | nd |
| 7 | K326 22 | 0.0162 | nd | nd | nd |
| 8 | K326 22 | 0.0500 | 0.0205 | nd | 0.0347 |
| 9 | K326 22 | 0.0204 | nd | nd | nd |
| 10 | K326 22 | 0.0138 | nd | nd | nd |
| | Average | 0.0218 | — | — | — |
| 11 | NCLA161 | 0.0081 | nd | nd | nd |
| 12 | NCLA161 | 0.0114 | nd | nd | nd |
| 13 | NCLA161 | 0.0075 | 0.0110 | nd | nd |
| 14 | NCLA161 | 0.0093 | nd | nd | nd |
| 15 | NCLA161 | 0.0072 | nd | nd | nd |
| | Average | 0.0087 | — | — | — |

These results demonstrate that the new hybrid cultivar NCLA161 of the present disclosure accumulates nicotine at levels below that of many other publicly reported cultivars. As described herein, naturally-occurring genetic variation and conventional breeding approaches were used to develop this new hybrid (i.e., gene editing, genetic engineering, or novel breeding approaches were not used to develop these materials). This hybrid can be grown anywhere in the world without concern regarding regulations pertaining to breeding outcomes of gene editing or genetic engineering. The tobacco hybrid, NCLA161, therefore offers commercial advantages of less regulatory burden as a non-transgenic, non-genetically engineered plant while being extraordinarily low in nicotine, nornicotine, anabasine and anatabine. In accordance with these data, embodiments of the present disclosure include an NCLA161 cultivar, or a cultivar derived therefrom.

### Example 2

A field study was conducted to assess the reduced levels of nicotinic alkaloids of the tobacco cultivars of the present disclosure. K326 and three backcross-derived low-alkaloid inbred lines were evaluated in two North Carolina field environments during the 2023 growing season. Experiments were conducted at the Upper Coastal Plain Research Station (Rocky Mount, NC) and the Oxford Tobacco Research Station (Oxford, NC). The experimental design used in each environment was a randomized complete block design with three replications. Plots consisted of single 20-plant rows and were managed according to standard flue-cured production practices for North Carolina. Intra- and inter-row spacing were 56 cm and 122 cm, respectively, at each location.

Tobacco plots were harvested at each of the two environments in four separate leaf harvests according to the rate of ripening and maturity. Leaf harvests were flue-cured and weighed. Fifty-gram composite samples of cured leaf were collected for each plot using a weighted mean basis. Oven-dried samples were ground and analyzed for alkaloid composition using gas chromatography, and the results are provided below in Table 2.

TABLE 2
Means for K326 and Backcross-Derived Low Alkaloid Lines Averaged Over Two 2023
North Carolina Field Environments. (LSD = “least significant difference.)

| | | | | | Total |
|---|---|---|---|---|---|
| | Nicotine | Nornicotine | Anatabine | Anabasine | Alkaloids |
| Genotype | (mg g⁻¹) | (mg g⁻¹) | (mg g⁻¹) | (mg g⁻¹) | (mg g⁻¹) |
| K326 | 26.263 | 1.019 | 0.747 | 0.155 | 28.184 |
| K326 (myc2a/myc2a) | 5.842 | 6.201 | 0.511 | 0.096 | 12.650 |
| K326 22 (nic1/nic1 | 2.961 | 0.098 | 0.099 | 0.020 | 3.178 |
| nic2/nic2) | | | | | |
| NCLA161 (K326 nic1/nic1 | 0.971 | 0.060 | 0.018 | 0.004 | 1.053 |
| nic2/nic2 myc2a/myc2a) | | | | | |
| LSD 0.05 | 5.155 | 1.244 | 0.140 | 0.026 | 5.314 |

Unless specified otherwise, analyte measurements of all nicotinic alkaloids (e.g., nicotine) were performed on the materials described herein on a dry weight basis. As would be recognized by one of ordinary skill in the art based on the present disclosure, levels of nicotine, nornicotine, anatabine, and anabasine can be measured by several methods known in the art. For example, nicotinic alkaloids can be quantified using gas chromatography (GC) and high-performance liquid chromatography. (See, e.g., Lisko et al 2013, Anal Chem. March 19; 85(6): 3380-3384, which provides methods for measuring quantities of nicotinic alkaloids in cigarette filters and in tobacco.) The analysis of tobacco alkaloids (e.g., nornicotine, anatabine, and anabasine) can also be performed with gas chromatography (GC) coupled with a wide spectrum of detection techniques, including but not limited to, flame ionization detection (FID), nitrogen-phosphorus detection (NPD), and mass spectrometry (MS). Other analytical approaches included high-performance liquid chromatography-ultraviolet detection (HPLC-UV), capillary zone electrophoresis-ultraviolet detection (CZE-UV), micellar electrokinetic capillary chromatography-ultraviolet detection (MECC-UV), nitrogen chemiluminescence detection (NCD), and microemulsion electrokinetic chromatography-ultraviolet detection (MEEKC-UV).

### Biological Deposits

Regenerative material of breeding line, NCLA161, respectively, is on deposit with the Crop and Soil Sciences Department, North Carolina State University, Raleigh, NC 27695. Specifically, NCLA161 is on deposit under accession number N23-303-2. A third-party expert, approved as such in advance by NCSU, can obtain a sample of the biological material, for either line, via written request to Ramsey S. Lewis, University Faculty Scholar and Charles and Marilyn Stuber Distinguished Professor of Plant Breeding, Campus Box 7620, Crop and Soil Sciences Department, North Carolina State University, Raleigh, NC 27695 (tel: (919)-513-4802).

Except as permitted under 37 CFR § 1.808(b), all restrictions imposed on the availability to the public of the deposited biological material will be irrevocably removed upon the granting of a patent from this application or any application citing it for purposes of priority.

What is claimed is:

1. A tobacco cultivar, or any part thereof, comprising reduced levels of at least one nicotinic alkaloid compared to a corresponding naturally-occurring tobacco plant, or part thereof.

2. The tobacco cultivar of claim 1, wherein the cultivar is non-transgenic.

3. The tobacco cultivar of claim 1 or claim 2, wherein the at least one nicotinic alkaloid is selected from the group consisting of nicotine, nornicotine, anatabine, and anabasine.

4. The tobacco cultivar of any of claims 1 to 3, wherein the at least one nicotinic alkaloid is nicotine, and wherein the cultivar comprises no more than 0.29% nicotine.

5. The tobacco cultivar of any of claims 1 to 4, wherein the at least one nicotinic alkaloid is nornicotine, and wherein the cultivar comprises no more than 0.02% nornicotine.

6. The tobacco cultivar of any of claims 1 to 5, wherein the at least one nicotinic alkaloid is anatabine, and wherein the cultivar comprises no more than 0.01% anatabine.

7. The tobacco cultivar of any of claims 1 to 6, wherein the at least one nicotinic alkaloid is anabasine, and wherein the cultivar comprises no more than 0.002% anabasine.

8. The tobacco cultivar of claim 1 or claim 2, wherein the at least one nicotinic alkaloid is nicotine and nornicotine, and wherein the cultivar comprises no more than 0.29% nicotine and no more than 0.02% nornicotine.

9. The tobacco cultivar of any of claims 1 to 8, wherein the cultivar comprises a nic1 allele having reduced expression and/or function compared to wildtype NIC1.

10. The tobacco cultivar of any of claims 1 to 9, wherein the cultivar comprises a nic2 allele having reduced expression and/or function compared to wildtype NIC2.

11. The tobacco cultivar of claim 9 or claim 10, wherein the nic1 and/or the nic2 allele is derived from at least one of the following lines: LAFC53, LAK326, LATN90, MAFC5, LMAFC34, LAMD 609, Lonibow, Vector 21-41, LA Burley 21, LI Burley 21, and HI Burley 21.

12. The tobacco cultivar of any of claims 1 to 8, wherein the cultivar comprises a myc2a allele having reduced expression and/or function compared to wildtype MYC2a.

13. The tobacco cultivar of claim 12, wherein the myc2a allele comprises at least one nucleotide deletion relative to a wild type MYC2a allele set forth in SEQ ID NO: 1.

14. The tobacco cultivar of claim 12, wherein the myc2a allele comprises a deletion of one to five nucleotides relative to a wild type MYC2a allele set forth in SEQ ID NO: 1.

15. The tobacco cultivar of claim 12, wherein the myc2a allele comprises a deletion of five nucleotides relative to a wild type MYC2a allele set forth in SEQ ID NO: 1.

16. The tobacco cultivar of any one of claims 13 to 15, wherein the deletion produces a truncated MYC2A protein.

17. The tobacco cultivar of any one of claims 12 to 16, wherein the myc2a allele comprises a nucleic acid sequence that is at least 70% identical to SEQ ID NO: 2.

18. The tobacco cultivar of any one of claims 12 to 16, wherein the myc2a allele comprises a nucleic acid sequence that is at least 80% identical to SEQ ID NO: 2.

19. The tobacco cultivar of any one of claims 12 to 16, wherein the myc2a allele comprises a nucleic acid sequence that is at least 90% identical to SEQ ID NO: 2.

20. The tobacco cultivar of any one of claims 12 to 19, wherein the myc2a allele is derived from N. tabacum.

21. The tobacco cultivar of any one of claims 9 to 20, wherein the cultivar further comprises suppression of expression within the cultivar of at least one of NBB1, A622, quinolate phosphoribosyltransferase (QPT), putrescine N-methyltransferase (PMT), ornithine decarboxylase (ODC), aspartate oxidase (AO), quinolinic acid synthase (QS), N-methylputrescine oxidase (MPO), NtERF221, NtMYC1a, NtMYC1b, or NtMYC2b.

22. The tobacco cultivar of any one of claims 1 to 22, wherein the cultivar is an NCLA161 cultivar, or derived therefrom.

23. A progeny plant, seed, or cell produced from any of the tobacco cultivars of claims 1 to 22.

24. A tobacco product derived from any of the tobacco cultivars, or parts therefrom, of claims 1 to 22.

25. The tobacco product of claim 24, wherein the product is selected from the group consisting of leaf tobacco, shredded tobacco, cut tobacco, ground tobacco, powder tobacco, reconstituted tobacco, tobacco extract, smokeless tobacco, moist or dry snuff, snus, pipe tobacco, cigar tobacco, cigarillo tobacco, cigarette tobacco, and chewing tobacco.

26. The tobacco product of claim 24, wherein the product is selected from the group consisting of a cigarillo, a cigarette, a kretek cigarette, a filter cigarette, a make-your-own cigarette, a roll-your-own cigarette, a stick or pod for a heated tobacco product, a cigar, snus, tobacco-containing gum, tobacco-containing lozenges, and chewing tobacco.

27. A method of producing a tobacco cultivar comprising reduced levels of at least one nicotinic alkaloid compared to a corresponding naturally-occurring tobacco plant, or part thereof, the method comprising:
crossing a first tobacco variety comprising a first low nicotine trait with a second tobacco variety comprising a second low nicotine trait to produce a progeny plant;
wherein the progeny plant comprises a reduced concentration of at least one nicotinic alkaloid, as compared to either the first or second tobacco variety.

28. The method of claim 27, wherein the method comprises backcrossing.

29. The method of claim 27 or claim 28, wherein the at least one nicotinic alkaloid is selected from the group consisting of nicotine, nornicotine, anatabine, and anabasine.

30. The method of any of claims 27 to 29, wherein the at least one nicotinic alkaloid is nicotine, and wherein the cultivar comprises no more than 0.29% nicotine.

31. The method of any of claims 27 to 30, wherein the at least one nicotinic alkaloid is nornicotine, and wherein the cultivar comprises no more than 0.02% nornicotine.

32. The method of any of claims 27 to 31, wherein the at least one nicotinic alkaloid is anatabine, and wherein the cultivar comprises no more than 0.01% anatabine.

33. The method of any of claims 27 to 32, wherein the at least one nicotinic alkaloid is anabasine, and wherein the cultivar comprises no more than 0.002% anabasine.

34. The method of any of claims 27 to 33, wherein the at least one nicotinic alkaloid is nicotine and nornicotine, and wherein the cultivar comprises no more than 0.29% nicotine and no more than 0.02% nornicotine.

35. The method of any of claims 27 to 34, wherein the first and/or second low nicotine trait comprises a nic1 allele having reduced expression and/or function compared to wildtype NICL.

36. The method of any of claims 27 to 35, wherein the first and/or second low nicotine trait comprises a nic2 allele having reduced expression and/or function compared to wildtype NIC2.

37. The method of any of claims 27 to 36, wherein the first and/or second tobacco variety comprises a nic1 and/or a nic2 allele derived from at least one of the following lines: LAFC53, LAK326, LATN90, MAFC5, LMAFC34, LAMD 609, Lonibow, Vector 21-41, LA Burley 21, LI Burley 21, and HI Burley 21.

38. The method of any of claims 27 to 37, wherein the first and/or second tobacco variety comprises a myc2a allele having reduced expression and/or function compared to wildtype MYC2a.

39. The method of claim 38, wherein the myc2a allele comprises at least one nucleotide deletion relative to a wild type MYC2a allele set forth in SEQ ID NO: 1.

40. The method of claim 39, wherein the myc2a allele comprises a deletion of one to five nucleotides relative to a wild type MYC2a allele set forth in SEQ ID NO: 1.

41. The method of claim 40, wherein the myc2a allele comprises a deletion of five nucleotides relative to a wild type MYC2a allele set forth in SEQ ID NO: 1.

42. The method of any one of claims 39 to 41, wherein the deletion produces a truncated MYC2A protein.

43. The method of any one of claims 39 to 42, wherein the myc2a allele comprises a nucleic acid sequence that is at least 70% identical to SEQ ID NO: 2.

44. The method of any one of claims 39 to 42, wherein the myc2a allele comprises a nucleic acid sequence that is at least 80% identical to SEQ ID NO: 2.

45. The method of any one of claims 39 to 42, wherein the myc2a allele comprises a nucleic acid sequence that is at least 90% identical to SEQ ID NO: 2.

46. The method of any one of claims 39 to 45, wherein the myc2a allele is derived from N. tabacum.

47. The method of any of claims 27 to 46, wherein the first low nicotine trait comprises a nic1 and/or nic2 allele having reduced expression and/or function compared to wildtype NIC1 and/or NIC2, and wherein the second low nicotine trait comprises a myc2a allele having reduced expression and/or function compared to wildtype MYC2a.

48. The method of any of claims 27 to 47, wherein the first tobacco variety, the second tobacco variety, and the progeny plant are non-transgenic.

49. A seed or cell obtained from the progeny plant produced according to the method of claim 48.

50. A tobacco product derived from the progeny plant produced according to the method of claim 48.

51. The tobacco product of claim 50, wherein the product is selected from the group consisting of leaf tobacco, shredded tobacco, cut tobacco, ground tobacco, powder tobacco, reconstituted tobacco, tobacco extract, smokeless tobacco, moist or dry snuff, snus, pipe tobacco, cigar tobacco, cigarillo tobacco, cigarette tobacco, and chewing tobacco.

52. The tobacco product of claim 50, wherein the product is selected from the group consisting of a cigarillo, a cigarette, a kretek cigarette, a filter cigarette, a make-your-own cigarette, a roll-your-own cigarette, a stick or pod for a heated tobacco product, a cigar, snus, tobacco-containing gum, tobacco-containing lozenges, and chewing tobacco.

53. A method of producing a Nicotiana tabacum plant having reduced nicotinic alkaloid content, the method comprising:
combining in a Nicotiana tabacum plant:
(a) one or more genetic modifications that reduces the expression and/or function of MYC2A; and
(b) a recessive allele of nic1 and/or a recessive allele of nic2;
wherein the Nicotiana tabacum plant has a nicotinic alkaloid content that is reduced as compared to a corresponding naturally-occurring or non-transformed control tobacco plant.

54. The method of claim 53, wherein the Nicotiana tabacum plant comprises a homozygous recessive allele of nic1 and/or a homozygous recessive allele of nic2.

55. The method of claim 53 or claim 54, wherein the one or more genetic modifications that reduces the expression and/or function of MYC2A is introduced by a Transcription activator-like effector nuclease (TALEN), meganuclease, zinc finger nuclease, a CRISPR/Cas9 system, a CRISPR/Cpf1 system, a CRISPR/Csm1 system, a gene knock-in technique or technology, or any combination thereof.

56. The method of any one of claims 53 to 55, further comprising suppressing expression within the Nicotiana tabacum plant at least one of NBB1, A622, quinolate. phosphoribosyltransferase (QP7), putrescine N-methyltransferase (PMT), ornithine decarboxylase (ODC), aspartate oxidase (AO), quinolinic acid synthase (QS), N-methylputrescine oxidase (MPO), NtERF221, NtMYC1a, NtMYC1b, or NtMYC2b.

57. A Nicotiana tabacum plant produced by the method of any one of claims 53 to 56, wherein the plant comprises: (a) one or more genetic modifications that reduces the expression and/or function of MYC2A; and (b) a recessive allele of nic1 and/or a recessive allele of nic2.

58. A progeny plant or seed produced from the plant of claim 57, wherein the progeny plant or seed comprises: (a) one or more genetic modifications that reduces the expression and/or function of MYC2A; and (b) a recessive allele of nic1 and/or a recessive allele of nic2.

59. A tobacco product comprising tobacco from the Nicotiana tabacum plant of claim 57, wherein the plant comprises: (a) one or more genetic modifications that reduces the expression and/or function of MYC2A; and (b) a recessive allele of nic1 and/or a recessive allele of nic2.

60. The tobacco product of claim 59, wherein:
(a) the tobacco is selected from the group consisting of leaf tobacco, shredded tobacco, cut tobacco, ground tobacco, powder tobacco, reconstituted tobacco, tobacco extract, smokeless tobacco, moist or dry snuff, snus, pipe tobacco, cigar tobacco, cigarillo tobacco, cigarette tobacco, and chewing tobacco; or
(b) the product is a reduced-nicotine tobacco product selected from the group consisting of a cigarillo, a cigarette, a kretek cigarette, a filter cigarette, a make-your-own cigarette, a roll-your-own cigarette, a stick or pod for a heated tobacco product, a cigar, snuff, snus, tobacco-containing gum, tobacco-containing lozenges, and chewing tobacco.
